(19) United States
(12) Patent Application Publication
(10) Pub. No.: US 2022/0403423 A1
(43) Pub. Date: Dec. 22, 2022

(54) INTEGRATED BIOREFINERY SYSTEM AND METHOD

(71) Applicant: University of Louisiana Lafayette, Lafayette, LA (US)

(72) Inventors: MARK E. ZAPPI, Lafayette, LA (US); RAFAEL HERNANDEZ, Lafayette, LA (US); DHAN LORD FORTELA, Lafayette, LA (US); PRASHANTH BUCHIREDDY, Lafayette, LA (US); EMMANUEL REVELLAME, Lafayette, LA (US); WAYNE SHARP, Lafayette, LA (US); JOHN GUILLORY, Lafayette, LA (US); DANIEL GANG, Lafayette, LA (US); WILLIAM HOLMES, Lafayette, LA (US)

(73) Assignee: University of Louisiana Lafayette, Lafayette, LA (US)

(21) Appl. No.: 17/846,505

(22) Filed: Jun. 22, 2022

Related U.S. Application Data
(60) Provisional application No. 63/213,521, filed on Jun. 22, 2021.

(51) Int. Cl. C12P 7/40; C01B 3/02; C10J 3/82; C11B 1/10; C02F 11/04; C10B 53/00; C10B 57/14; B09B 3/40; B09B 3/65; C10B 53/02

(52) U.S. Cl. CPC C12P 7/40; C01B 3/02; C10J 3/82; C11B 1/10; C02F 11/04; C10B 53/00; C10B 57/14; B09B 3/40; B09B 3/65; C10B 53/02; C10J 2300/0946; C10J 2300/1625; C10J 2300/1656; B09B 2101/75

(57) ABSTRACT

The inventive biorefinery system and method accepts municipal solid waste, sewage sludges, and/or ag-wastes and processes it through three primary conversion unit operations to produce a variety of value-added products. In a preferred embodiment, the three primary conversion units are gasification, thermal depolymerization or torrefaction/pyrolysis, and biotreatment.

### CROSS-REFERENCE TO RELATED APPLICATIONS

This application claims priority to U.S. Provisional Application No. 63/213,521 entitled “Integrated Biorefinery System and Method” and filed on Jun. 22, 2021.

### STATEMENT REGARDING FEDERALLY SPONSORED RESARCH OR DEVELOPMENT

Not Applicable

### BACKGROUND OF THE INVENTION

### Field of the Invention

This invention relates to the field of waste management. In particular, the invention relates to the management of municipal solid waste, sewage sludge, and agricultural waste.

### Background Information

Management and disposal of municipal solid waste (“MSW”) and sewage sludge (SS) generated within urban and suburban regions continues to challenge the ability to resource-support these fast-growing areas of dense human populations and industrial activities. This issue is considered by most experts to be a global crisis. The US produces over 7 million tons of biosolids every year at wastewater treatment plants (“WWTPs”). The vast majority of these WWTPs are activated sludge biotreatment systems coupled with some form of waste sludge digestion. Either anaerobic or aerobic digestion (“AD”) is used for sludge stabilization with anaerobic digestion being more popular. Three types of sludges are generated at WWTPs (primary sludge, secondary sludge, & biosolids). Primary sludge and waste activated sludge (“WAS”) are made up of settled raw sewage solids and wasted aerobic bacteria, respectively. Both are fed into the digesters where they are biologically degraded into a final sludge that is dewatered to about 15-25% dry solids to become “biosolids”. At most WWTP, biosolids are the only waste sludge exiting the plant because both primary sludge and WAS are usually digested. Biosolids are 40% landfilled, 40% land-applied, 10% incinerated, and 10% used as fertilizer.

During AD, biogas is produced which provides a medium grade fuel (approximately 700 BTUs/cf at 70% CH4). The Water Environment Federation (professional industry organization for WWTPs) has urged performance of R&D on biosolids to truly develop them into a feedstock to biorefineries.

WWTP sludges are approximately 65-80% thermally reactive with WAS being approximately 80% biodegradable and biosolids approximately 50% biodegradable. In 2015, over 260 million tons of MSW were generated in the US, of which 52.5% was landfilled, 25.8% was recycled, 12.8% was burned with energy recovery, and 8.9% was composted. MSW is approximately 80% thermally reactive (combustible) and approximately 60% biodegradable. Over 500 million tons of construction/demolition material is produced each year within urban areas of the US. Of this tonnage, over 50 million tons/year are thermally reactive. When added to the EPA-defined MSW, this comes to over 300 million tons/year of available materials in the US.

In the US, very little MSW is used for energy production (13%). About 30% of the US MSW is recycled with the bulk of this tonnage being glass, metals, and some paper. This means that the bulk of this tonnage is not thermally reactive or bio-degradable and thus not viable energy or chemical feedstocks. Accordingly, the bulk of MSW disposed in landfills are thermally reactive and/or biodegradable.

The American Society of Civil Engineers (“ASCE”) in its 2017 Infrastructure Report Card requested that the US Government invest in addressing a comprehensive MSW conversion system(s) that would use MSW as feedstocks and result in new products. ASCE encourage leaders to rethink MSW as a resource. This is a definitive example of the need for and novelty of this invention.

The costs to dispose of MSW and SS wastes is approximately $30-$200/ton. Additionally, almost 1 billion tons per year of animal manures are produced within the US. Examples include manures from poultry, dairy, and swine raising operations. If only 10% of the animal manures are included, the combined total waste feedstock within the US each year exceeds over 420 million tons/year which represents a vast, relatively untapped renewable feedstock that can be used to produce commercially valuable energy fuels and co-products while solving several significant disposal problems that are only growing in scope and size.

Almost all biorefineries proposed/designed/tested to date within the US have been single conversion process-based systems. That is, gasification, torrefaction, pyrolysis, hydrothermal conversion, fermentation, or anaerobic digestion were individually evaluated for their ability to serve as “biorefineries” to convert biomass into mainly biofuels with some producing non-fuel bioproducts. Single mechanism conversion is not the methodology used in petroleum refineries where numerous staged unit operations are combined to form conversion pathways through the different conversion stages depending on feedstock composition.

Single unit operation biorefinery systems have been proven technically (perhaps not fully economically in many cases) viable for ag-waste and even some with SS where these materials are generally homogeneous in terms of compositional content and chemical complexity (relatively simple compared to MSW).

However, much like petroleum refineries handle different crudes, an urban waste-fed biorefinery must have processing flexibility via multiple conversion paths to manage different feedstock fractions. This invention represents a biorefinery system and method with the process flexibility to convert combinations of MSW, SS, and/or ag-residuals, into value added products. These feedstock blends represent the bulk of urban and suburban wastes.

The benefits of the inventive biorefinery system and method over the other single conversion systems used in the past is that a much larger portion of the input wastes will be converted into as many products instead of only one or two with the past designs.

### SUMMARY OF THE INVENTION

In one or more embodiments, this invention represents a biorefinery system and method with the process flexibility to convert combinations of MSW, SS, and/or ag-residuals, into value added products. These feedstock blends represent the bulk of urban and suburban wastes.

The system may comprise segregating a municipal solid waste feedstock resulting in combustible feedstock, complex organic feedstock, and bioconvertible feedstock; gasifying said combustible feedstock to produce syngas; fermenting at least a portion of said produced syngas to create residual acetic acid; thermal processing said complex organic feedstock to produce biochar, biocoal and/or biooil or biocrude; and biotreating said residual acetic acid to form waste activated sludge containing lipids.

### BRIEF DESCRIPTION OF THE DRAWINGS

The drawings constitute a part of this specification and include exemplary embodiments of the Integrated Biorefinery System and Method which may be embodied in various forms. It is to be understood that in some instances, various aspects of the invention may be shown exaggerated or enlarged to facilitate an understanding of the invention. Therefore, the drawings may not be to scale

FIG. 1 is a process flow diagram depiction of the inventive biorefinery system and method.

FIG. 2 is a process flow diagram depiction of the feedstock.

### DETAILED DESCRIPTION OF THE INVENTION

The subject matter of the present invention is described with specificity herein to meet statutory requirements. However, the description itself is not intended to necessarily limit the scope of claims.

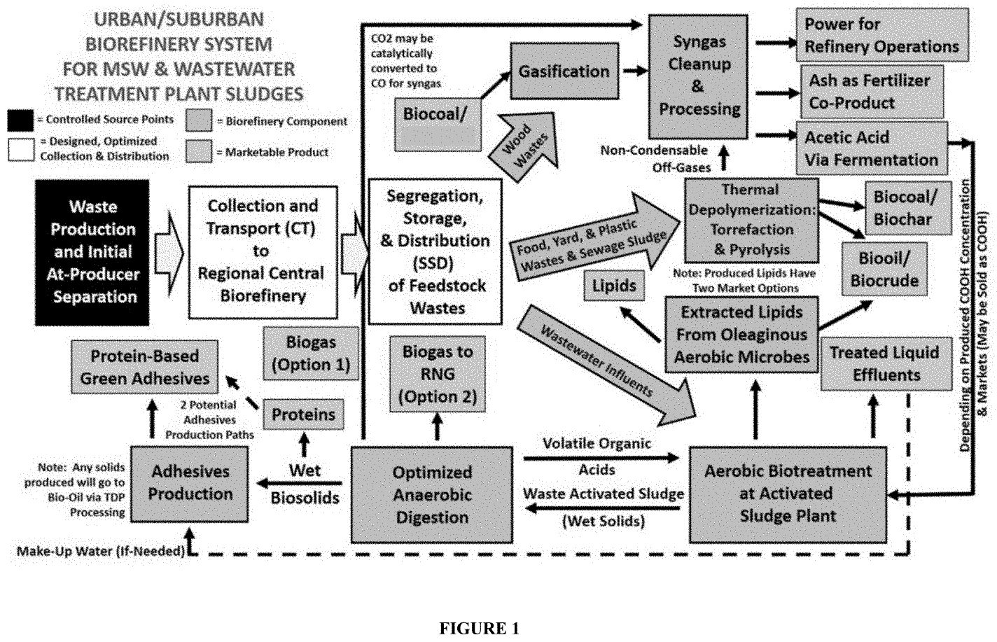

A comprehensive bio-refinery process for urban and suburban wastes is claimed herein that consists of individual unit operations that have been proven effective for use with primarily agricultural feedstocks and, to a lesser degree, MSW and SS. The state of technology is composed of proven concepts for homogeneous biomass feedstocks. However, the integration and use of urban/suburban wastes are novel—particularly when using the wide variety of urban waste feedstocks/inputs available in the forms of MSW, SS, and ag-residuals. The system and method (summarized in FIG. 1) is unique in terms of application and level of integration. The performance targets are >70% carbon conversion to products and limited emissions (via Life Cycle Assessment analysis), profitable income streams (via techno-economic assessment), and >90% reduction of current disposal costs (shift from cost to profit).

In one or more embodiments, the biorefinery system and method has the following key unit operations/processing steps embedded in the design:

### Feedstock Management

Feedstock collection, transport, segregation, storage, and distribution (“CTSSD”) is key to the success of any system where urban/suburban wastes are to serve as an industrial feedstock. Past work with recycling and attempted processing of MSW failed (more than sewage sludge that is homogeneous and single point accessible), often at this stage. This inventive biorefinery system and method optimizes point separation, collection, centralized segregation, storage (needed for pre & post-segregation), and process feed systems while keeping costs and odor issues down.

### Gasification

Gasification is the thermal conversion of combustible feedstocks into synthesis gas or syngas. The produced syngas can be used as a gaseous fuel (˜200 BTU/scf) or as a feedstock for catalytic or biological conversion into a variety of fuels and chemicals including but not limited to ethanol, acetic acid, gasoline, aviation fuel, hydrogen, and biocrude. In one or more embodiments, the products from gasification comprise, syngas (chemicals and fuels including hydrogen, power generation & heat), acetic acid, and ash fertilizer. In other embodiments, the inventive biorefinery system and method can be modified to produce a bio-oil (biocrude). Any suitable feedstock may be used. In one embodiment, woody wastes which have been dried are used for feedstock, and/or for both gasification and TDP (Torrefaction & Pyrolysis).

### Thermal Depolymerization or Torrefaction, Hydrothermal Conversion, and Pyrolysis

The next conversion process within the inventive biorefinery system and method is thermal depolymerization which is defined herein as a class of thermal processes that use thermal energy to partially breakdown the molecular bonds of complex organic materials such as MSW and SS into biocoal and/or bio-oils, including biocrude. Biocrude can be further processed into a variety of chemicals and fuels. The reactors for the thermal depolymerization are selected based on the capacity of the WWTP.

Torrefaction, hydrothermal conversion, and pyrolysis are known thermochemical processes that can be applied to waste feedstock to convert the feedstock into intermediate or final products. Torrefaction is the process by which feedstock is “charred” to produce biocoal in the zero or limited oxygen environment. Specifically, it is a thermochemical process that decreases the water content and other volatile compounds within the feedstock, which leads to an increase in energy density of the feedstock. The biocoal may then be used for power or heat production via combustion or be used as feedstock in the gasification process. The charring process generally operates between 200 to 400 degrees Celsius. The feedstock must be relatively dry. Pyrolysis on the other hand operates at higher temperatures and anaerobically. This results in a greater amount of liquid-bio-oils in addition to biochar-solids (which can be used as an agriculture amendment, as thermal fuel, or as a filler in structural, epoxy, and polymer composites). The feedstock must be relatively dry for torrefaction and pyrolysis. Whereas hydrothermal conversion can handle/process very high moisture and ash content feedstock to produce biooil and/or biochar or biocoal. The process takes place in a similar operational conditions as torrefaction but under higher pressures (20-150 bar).

### Biotreatment

The process comprises an aerobic biotreatment step followed by waste sludge digestion to produce biosolids at a solids content of approximately 20% after. This conversion option embodiment is generally used with high water content, bio-convertible feedstocks, including waste preprocessed to be bio-converted. Activated sludge/AD processes which represent the heart of most municipal and industrial WWTPs, are used in the preferred embodiment. Such essentially converts influent carbon into microbial cells and related products (biogas, lipids, or proteins).

AD converts the influent organics into organic acids—via acidogens then to acetogens—and then to biogas. The resulting biogas is mainly methane and carbon dioxide (via methanogens). Volatile organic acids (“VOAs”), such as acetic, butyric, lactic, and propionic acids, are the key intermediates prior to biogas production.

### Process Description

The inventive biorefinery system and method accepts MSW, sewage sludges, and/or ag-wastes and processes it through three primary conversion unit operations (gasification, TDP, and biotreatment) to produce a variety of products. Such products comprise: biocrude to replace crude oil in refineries, biocoal to offer a green alternative to fossil-based coal, fats and oils to be used as animal feed supplements and as a feed into producing bio-based fuels, electrical power, ash fertilizer, proteins for use as an animal feed supplement, biogas used as a replacement to natural gas, biohydrogen, recovered treated water, electrical power, acetic acid to be used as an green industrial feedstock, and glues made from sewage sludges. The inventive biorefinery system and method may also produce heat and steam capable of being captured as a recovered resource.

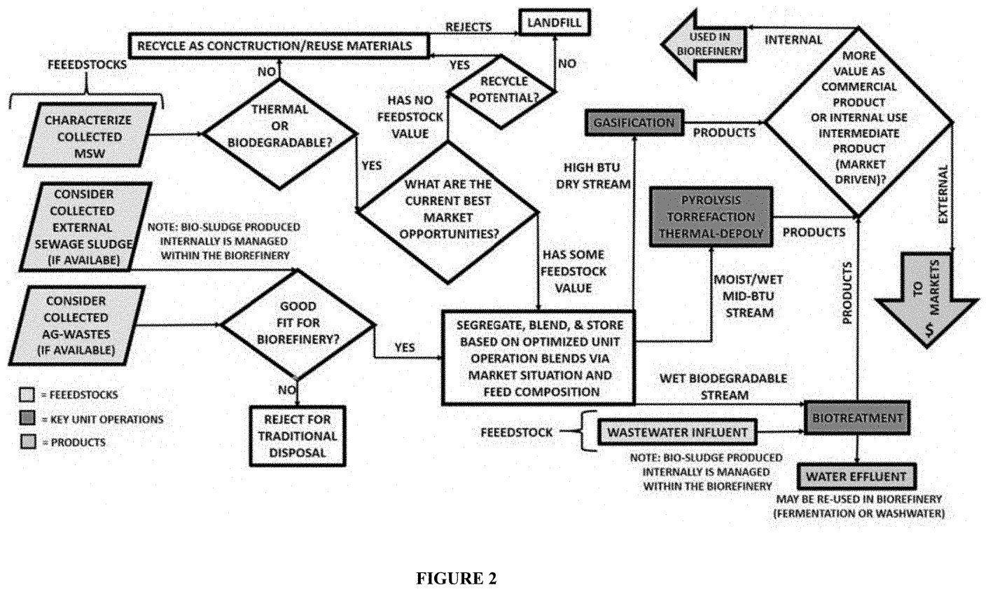

The three conversion unit operations provide flexibility to handle a high percentage of the urban and suburban wastes. FIG. 2 provides one embodiment of a feedstock processing flow-path logic system throughout the inventive biorefinery system and method based on the feedstock characteristics and selected feed mass-flow distribution across a three unit operations.

The inventive biorefinery system and method influents include wastewater from the urban/suburban populace and industrial inputs as activated sludge. These influents are passed through the biotreatment stage. Then, the resulting materials are processed through anaerobic digestion. Lipids are formed within the cells of the aerobes within the activated sludge process through the conversion of the influent organic fractions, the VOAs produced in the AD system, and in various embodiments, the water from the TDP unit operations.

Several embodiments may be used to enhance the rate, type, and amount of lipids that build-up within the cells of the aerobes. For example, introduction of oleaginous microbial seeds, VOA speciation selective feeding, nutrient starvation, SRT, concentrated VOA feeds, and thermal shocking may all be used separately or in some combination thereof. The SS generated can be used to produce adhesives, biogas, and proteins or potentially be fed into the thermal conversion units within the biorefinery.

In an exemplary embodiment, the system comprise segregating a municipal solid waste feedstock resulting in combustible feedstock, complex organic feedstock, and bio convertible feedstock; gasifying said combustible feedstock to produce syngas; fermenting at least a portion of said produced syngas to create residual acetic acid; thermal processing said complex organic feedstock to produce biocrude, biocoal, and/or biochar; and biotreating said residual acetic acid to form waste activated sludge containing lipids. The thermal processing step may be torrefaction, hydrothermal conversion, and/or pyrolysis.

The system may further include the step of optimizing the anaerobic digestion of said activated sludge to form wet biosolids. The system may also include the biotreating step further comprises the steps of extracting lipids from oleaginous aerobic microbes to create biooil and lipids. The system may also include a portion of said syngas of said gasifying step being catalyzed to produce at least one chemical (the chemical may include primary alcohols, gasoline, diesel, hydrogen, and at least one chemical may be used for power generation).

Feedstock for the system may include municipal solid waste comprising a plastic, lignocellulosic, food, or biosludge component and wherein said thermal processing step comprises depolymerization of said plastic, lignocellulosic, food, or biosludge component of said municipal solid waste.

The inventive biorefinery system and method may be used for a variety of waste conversion applications, including: to refine agricultural manures, agricultural residuals, crops, meat processing residuals, lignocellulosic materials, waste wood, algae, fermentation residuals, medical wastes, industrial wastes, waste tires, demolition wastes, and spent adsorbents into the above stated products.

The product moves through the system as follows and in accordance with FIG. 2. Collected MSW is sorted by its ability to be thermally or biologically degraded. If the MSW cannot be degraded, it is sent for potential recycling. If the MSW is degradable and can be used as a feedstock, it is segregated, blended, and stored based on optimization of the unit processes. Similarly, sewage sludge and agriculture waste that are suited for biorefinery are is segregated, blended, and stored.

The suitable feedstock is then sent to the biotreatment unit if it has a high moisture content. The biotreatment unit may also have wastewater influent. Biotreatment may then result in water effluent that can be re-used in the biorefinery system in the fermentation process or as wash water.

The suitable feedstock that has a high btu and relatively dry is sent to the gasification unit. The suitable feedstock that has a high moisture content and mid-level btu is sent to the thermal treatment units. Both units result in commercial value products that can be sent to market or re-used in the biorefinery system as previously discussed.

An exemplary embodiment is as follows: A biorefinery method comprising: segregating a municipal solid waste feedstock resulting in combustible feedstock, complex organic feedstock, and bioconvertible feedstock; gasifying said combustible feedstock to produce syngas; fermenting at least a portion of said produced syngas to create residual carboxylic acids; thermal processing said complex organic feedstock to produce biochar, biocrude, or biocoal; and biotreating said residual carboxyl acids to form waste activated sludge containing lipids. The method described wherein said complex organic feedstock is capable of being torrefied into one of the following: biocoal, biochar, biooil, and carbon black. The method described wherein said complex organic feedstock is capable of being thermally converted into one of the following: biocoal, biochar, biooil, and carbon black. The method of described wherein in the gasifying step, said combustible feedstock is gasified to produce green hydrogen. The method of described further comprising the step of optimizing anaerobic digestion of said activated sludge to form wet biosolids. The method described wherein said biotreating step further comprises the steps of extracting lipids from oleaginous aerobic microbes to create biooil and lipids. The method of described wherein said biotreating step further comprises the steps of extracting lipids from oleaginous anaerobic microbes to create biooil and lipids. The method described wherein said municipal solid waste comprises a plastic, lignocellulosic, food, or biosludge component and wherein said thermal processing step comprises depolymerization of said plastic, lignocellulosic, food, or biosludge component of said municipal solid waste. The method described wherein a portion of said syngas of said gasifying step is catalyzed to produce at least one chemical. The method described wherein said at least one chemical is selected from the group consisting of primary alcohols, gasoline, diesel, hydrogen, and wherein said at least one chemical is used for power generation. The described wherein said thermal processing step comprises torrefaction. The method described wherein said thermal processing step comprises pyrolysis. The method described wherein said thermal processing step comprises hydrothermal conversion. The method described wherein said biotreating comprises aerobic biotreatment. The method described wherein said produced syngas comprises hydrogen and further comprising the step of segregating at least a portion of said hydrogen. The method described wherein said produced syngas comprises hydrogen and further comprising the step of catalytically converting at least a portion of said hydrogen. The method described wherein at least a portion of said produced syngas is used in power protection through combustion of said produced syngas. The method described further comprising the step of anaerobic digestion of said activated sludge and sewage sludge forming biogas for conversion to hydrogen. The method of described further comprising the step of anaerobic digestion of said activated sludge and sewage sludge forming biogas for conversion to natural gas.

For the purpose of understanding the INTEGRATED BIOREFINERY SYSTEM AND METHOD references are made in the text to exemplary embodiments of an INTEGRATED BIOREFINERY SYSTEM AND METHOD, only some of which are described herein. It should be understood that no limitations on the scope of the invention are intended by describing these exemplary embodiments. One of ordinary skill in the art will readily appreciate that alternate but functionally equivalent components, materials, designs, and equipment may be used. The inclusion of additional elements may be deemed readily apparent and obvious to one of ordinary skill in the art. Specific elements disclosed herein are not to be interpreted as limiting, but rather as a basis for the claims and as a representative basis for teaching one of ordinary skill in the art to employ the present invention.

What is claimed is:

1. A biorefinery method comprising:
a. segregating a municipal solid waste feedstock resulting in combustible feedstock, complex organic feedstock, and bioconvertible feedstock;
b. gasifying said combustible feedstock to produce syngas;
c. fermenting at least a portion of said produced syngas to create residual carboxylic acids;
d. thermal processing said complex organic feedstock to produce biochar, biocrude, or biocoal; and
e. biotreating said residual carboxyl acids to form waste activated sludge containing lipids.

2. The method of claim 1 wherein said complex organic feedstock is capable of being torrefied into one of the following: biocoal, biochar, biooil, and carbon black.

3. The method of claim 1 wherein said complex organic feedstock is capable of being thermally converted into one of the following: biocoal, biochar, biooil, and carbon black.

4. The method of claim 1 wherein in the gasifying step, said combustible feedstock is gasified to produce green hydrogen.

5. The method of claim 1 further comprising the step of optimizing anaerobic digestion of said activated sludge to form wet biosolids.

6. The method of claim 1 wherein said biotreating step further comprises the steps of extracting lipids from oleaginous aerobic microbes to create biooil and lipids.

7. The method of claim 1 wherein said biotreating step further comprises the steps of extracting lipids from oleaginous anaerobic microbes to create biooil and lipids.

8. The method of claim 1 wherein said municipal solid waste comprises a plastic, lignocellulosic, food, or biosludge component and wherein said thermal processing step comprises depolymerization of said plastic, lignocellulosic, food, or biosludge component of said municipal solid waste.

9. The method of claim 1 wherein a portion of said syngas of said gasifying step is catalyzed to produce at least one chemical.

10. The method of claim 9 wherein said at least one chemical is selected from the group consisting of primary alcohols, gasoline, diesel, hydrogen, and wherein said at least one chemical is used for power generation.

11. The method of claim 1 wherein said thermal processing step comprises torrefaction.

12. The method of claim 1 wherein said thermal processing step comprises pyrolysis.

13. The method of claim 1 wherein said thermal processing step comprises hydrothermal conversion.

14. The method of claim 1 wherein said biotreating comprises aerobic biotreatment.

15. The method of claim 1 wherein said produced syngas comprises hydrogen and further comprising the step of segregating at least a portion of said hydrogen.

16. The method of claim 1 wherein said produced syngas comprises hydrogen and further comprising the step of catalytically converting at least a portion of said hydrogen.

17. The method of claim 1 further comprising the step of anaerobic digestion of said activated sludge and sewage sludge forming biogas for conversion to hydrogen.

18. The method of claim 1 further comprising the step of anaerobic digestion of said activated sludge and sewage sludge forming biogas for conversion to natural gas.
